(19) United States
(12) Patent Application Publication
(10) Pub. No.: US 2020/0121747 A1
(43) Pub. Date: Apr. 23, 2020

(54) COMPOSITION FOR PREVENTING OR TREATING ARTHRITIS COMPRISING HYDROQUINOIC ACID

(71) Applicant: PUKYONG NATIONAL UNIVERSITY INDUSTRY-UNIVERSITY COOPERATION FOUNDATION, Busan (KR)

(72) Inventor: Hyeung-Rak Kim, Busan (KR)

(73) Assignee: PUKYONG NATIONAL UNIVERSITY INDUSTRY-UNIVERSITY COOPERATION FOUNDATION, Busan (KR)

(21) Appl. No.: 16/176,823

(22) Filed: Oct. 31, 2018

(30) Foreign Application Priority Data
Oct. 23, 2018 (KR) 10-2018-0126775

(51) Int. Cl. A61K 36/02; A61P 19/02; A23L 33/105; A61K 31/065

(52) U.S. Cl. CPC A61K 36/02; A61P 19/02; A61K 2236/39; A61K 31/065; A61K 2236/35; A23L 33/105

(57) ABSTRACT

The present disclosure relates to a pharmaceutical composition for the prevention or treatment of arthritis, the composition including a sargahydroquinoic acid as an active ingredient. The sargahydroquinoic acid according to the present disclosure has an excellent activity to reduce or inhibit the activity of IL-1β, IL-6, or TNF-α, which is an inflammatory cytokine, and has an excellent effect of ameliorating arthritis in an arthritis animal model, and thus can be advantageously used as a composition for the treatment of arthritis. In addition, the sargahydroquinoic acid causes neither drug toxicity and side effects, and thus can be safely used even when taken for a long period of time, and has a stable effect even in the body.

### CROSS-REFERENCE TO RELATED APPLICATIONS

This application is based on and claims priority from Korean Patent Application No. 10-2018-0126775, filed on Oct. 23, 2018, with the Korean Intellectual Property Office, the disclosure of which is incorporated herein in its entirety by reference.

### TECHNICAL FIELD

The present disclosure relates to a composition for preventing or treating arthritis, in which the composition includes a sargahydroquinoic acid isolated from an extract of Sargassum serratifolium as an active ingredient.

### BACKGROUND

The human body is made of about 200 joints. The bones meet each other in the joints, which are composed of cartilage, joint capsule, synovial membrane, ligament, tendon, muscle, etc. in order to smoothly move between the bones and play a role in absorbing the impact caused by the movement.

Inflammatory diseases that occur in these joints can be divided largely into chronic rheumatoid arthritis, which is understood to be caused by autoimmunity, infectious arthritis caused by bacterial infection, deformed arthritis caused by degeneration or destruction of articular cartilage or bone due to various causes, and crystalline arthritis caused by deposition of soluble metabolites as crystals in the connective tissue around the joints due to degenerative changes of the connective tissue.

Degenerative arthritis, that is, osteoarthritis, is a disease caused by the destruction of joint tissue due to the increase of synthesis and activity of matrix metalloproteinase (MMP), which degrades the joint matrix in joint cells by the production of inflammatory cytokines, such as, interleukin-1β and tumor necrosis factor-α, while the degeneration of chondrocytes forming joints occurs due to aging, thereby inhibiting the synthesis of type II collagen and proteoglycan, which are matrix materials of joints in chondrocytes.

Further, the arthritis is worsened by the production of nitric oxide because of inflammatory cytokines and by the production of self-amplifying cytokines due to the produced nitric oxide, which leads to the synthesis of more MMPs and promotes the degradation of the joint matrix. At the same time, inflammatory cytokines increase the production of prostaglandin E2 that is a lipid metabolite, and thus, lead to an inflammatory response in arthritis.

Various biochemical phenomena are involved in the inflammatory reaction in vivo. Particularly, the inflammatory reaction is initiated or adjusted by various enzymes, which are related to the inflammatory response produced by immune cells. Specifically, the immune cells migrate to the injured site through the blood vessels with the help of histamine, nitric oxide (NO), prostaglandin E2 (PGE2), etc., and then, begin the inflammatory reaction. The immune cells that migrate to the injured site secrete cytokines such as tumor necrosis factor-α (TNF-α), interleukin-1β (IL-1β), or interleukin-6 (IL-6) or chemokines such as MIP-1, IL-8, or MCP-1 to destroy direct external invaders or collect other immune cells, thereby initiating the inflammatory response.

In the case where being exposed to inflammation-inducing substances such as interferon-λ, lipoteichoic acid, and lipopolysaccharide (LPS), which induce the inflammatory reaction, or various inflammation-inducing cytokines, inducible nitric oxide synthase (iNOS) and cyclooxygenase-2(COX-2) are expressed, thereby producing the excess NO and PGE2. The several inflammatory initiators (iNOS, COX-2, TNF-α, IL-6, etc.) promote transcription by activated NF-κB, so that NO is produced more than is needed, thereby causing vasodilation due to shock, tissue damage caused by an inflammatory response, mutagenesis, damage of nerve tissue, and the like.

Nitric oxide (NO) is produced from L-arginine and molecular oxygen by NO synthase (NOS). In mammals, there are three types of NOS: neuronal NOS (nNOS), endothelial NOS (eNOS), and inducible NOS (iNOS). nNOS and eNOS are constitutively expressed in nerve cells and endothelial cells. However, iNOS is inductively produced in macrophages or monocytes by exposure to LPS-induced or pro-inflammatory cytokines (Vane et al., 1994). NO produced in iNOS causes inflammation or an immune response, and thus acts as a proliferation inhibitor or a cytotoxic agent of a pathogen that invades the cell. However, it is known that the excess NO produced by overexpression of iNOS causes pathological conditions (Kim et al., 2005; Pan et al., 2011). The deleterious effect of excess NO in the cell not only acts as an inflammatory mediator itself, but also reacts with superoxide to produce peroxynitrite. Peroxynitrite may not only cause oxidative damage of intracellular molecules such as proteins, fats and DNA, but may also modify normal gene regulation. Thus, the overexpression of iNOS as well as large amounts of NO is closely related to pathological conditions associated with various inflammatory diseases (MacMicking et al., 1997; Maeda and Akaike, 1998).

Nuclear factor kappa B (NF-κB) plays a major role in immune and acute inflammatory responses as well as cell growth (Li and Verma, 2002; Makarov, 2001). NF-κB activation promotes the expressions of iNOS and several pro-inflammatory genes (Kim et al., 2005; Makarov, 2001). The activation pathway of NF-κB is such that the inhibitor of κB (IκB)-α kinase is phosphorylated by LPS followed by phosphorylation of IκB-α, and the IκB-α phosphorylated by ubiquitin is degraded to cause free NF-κB to move the nucleus, thereby adjusting the expression of inflammatory-related genes (Chen et al., 1995). Activation of another NF-κB occurs via mitogen-activated protein kinases (MAPKs) (Guha and Mackman, 2001) or phosphatidylinositol 3-kinase (PI3K)/protein kinase B (Akt) pathway (Sheu et al., 2005). MAPKs have extracellular signal-regulated kinases (ERKs), c-Jun NH2-terminal kinases (JNKs), and p38 MAPKs, which are involved in transcriptional regulation of inflammatory genes through NF-κB activation (Bhat et al., 1998; Kao et al., 2005; Shin et al., 2010). PI3K is also involved in the production of inflammatory cytokines through NF-κB activation (Cremer et al., 2011; Sheu et al., 2005). PI3K activation phosphorylates the phosphatidylinositide to activate the Akt protein. The activated PI3K/Akt plays a major role in the activation of macrophages (MacMicking et al., 1997; Sheu et al., 2005). Therefore, in order to develop an anti-inflammatory agent, many studies have been carried out to find a substance that inhibits the activation of NK-κB or inhibits the activation of MAPKs and Akt which activate NF-κB.

MMPs are proteolytic enzymes that destroy bone and cartilage matrix components and are expressed in cartilage tissues stimulated by inflammatory cytokines in inflammatory disease states, resulting in increased activity. MMPs constitute at least 21 enzymes, being classified into subclasses including collagenase (MMP-1, 8, and 13), stromelysin (MMP-3, 10, and 11), gelatinase (MMP-2 and 9), and matrix type-1 metalloproteinase (MMP-14). Among them, MMP-2 and MMP-9 are gelatinase subfamily, which is an important enzyme for collagen degradation of cartilage tissue. These two enzymes break down other substrates, such as fibrous collagen I and II, which are abundant in cartilage, and aggrecan.

Rheumatoid and degenerative arthritis are characterized by inflammatory cell infiltration into synovial tissue, which is mediated by chemokines. It is known that chemokines such as monocyte chemoattractant protein-1 (MCP-1) are expressed in the synovial tissue, and they are produced in synovial fibroblasts and the like. Excessive MCP-1, produced by arthritis, causes monocytes and macrophages to come in inflammatory sites and activates these cells to promote the production of inflammatory cytokines, thereby further aggravating the inflammation.

The expression of adhesion molecules such as vascular cellular adhesion molecule 1 (VCAM-1), intercellular adhesion molecule 1 (ICAM-1), and E-selectin in vascular endothelial cells is increased by cytokines in synovial tissues of rheumatoid and degenerative arthritis to induce infiltration of inflammatory cells. Overexpressed ICAM-1 and VCAM-1 are associated with chronic inflammation such as rheumatoid arthritis and degenerative arthritis.

Rheumatoid and degenerative arthritis are chronic systemic inflammatory diseases that cause symmetrical, multiple arthritis, resulting in joint damage and deformation. In the absence of treatment for these types of arthritis, the progression is poor, indicating a disability of the joint function, and if persistent, the disability of the joint function interferes with daily life. In Korea, it is estimated that about 1% of the total population is suffering from rheumatoid arthritis. It is known that the incidence ratio of rheumatoid arthritis is three times higher in women than in men, and it occurs mainly in the 20s to 40s.

The main causes of rheumatoid arthritis have been increasingly revealed, and genetic factors, infections, hormonal abnormalities, etc. are thought to be causative factors. Because of these causative factors, “autoimmune” phenomenon occurs. The autoimmune is a phenomenon that chronic inflammation occurs multiply and lastingly in many parts of the body due to abnormality of immune regulation function of our body.

Meanwhile, drugs used in the treatment of arthritis can be categorized on the basis of the main mechanism of action such as reduction of inflammation, delay of disease progression, and decrease of uric acid concentration. Many neuroarthritis treatment drugs act to reduce inflammation. Inflammation is a pathological process that causes pain, swelling, fever, seizures, and stiffness. Drugs that rapidly relieve inflammation include nonsteroidal anti-inflammatory drugs including aspirin and steroidal anti-inflammatory drugs including cortisone.

Nonsteroidal anti-inflammatory drugs have the effect of relaxing nerve joints and relieving inflammation by reduction of pain. However, because gastrointestinal disorders may occur or abdominal pain may be induced, use thereof is prohibited for some people with active peptic ulcer or hemorrhagic lesions in the gastrointestinal portion. Steroidal anti-inflammatory drugs are not used well for degenerative arthritis due to severe side effects such as weight gain and hypertension compared to their effects.

In particular, steroidal anti-inflammatory drugs have nothing to do with the causative treatment of the disease, and may simply induce excessive use of joints by temporarily reducing the pain, which causes destruction of the nerve joints and deterioration of the disorder so that it requires attention in use.

Therefore, conventional therapies used for joint damage such as arthritis have limited effectiveness, involve obvious toxic side effects, cannot be used continuously for a long period of time, and thus their effectiveness is limited. Thus, there is a desperate need for a novel therapeutic method or a therapeutic agent that overcomes the disadvantages of conventional therapeutic methods.

### RELATED ART DOCUMENT

### Patent Document

(Patent Document 1): Korean Patent Registration No. 10-1745338

### SUMMARY

Accordingly, the inventors of the present disclosure have found that a sargahydroquinoic acid isolated from an extract of Sargassum serratifolium has an effect of preventing and treating arthritis, thus completing the present disclosure.

Accordingly, the present disclosure provides a pharmaceutical composition and a health functional food for preventing or treating arthritis, in which the pharmaceutical composition includes a sargahydroquinoic acid as an active ingredient.

In order to achieve the above objects, the present disclosure provides a pharmaceutical composition for preventing or treating arthritis, in which the pharmaceutical composition includes a sargahydroquinoic acid as an active ingredient.

In addition, the present disclosure provides a health functional food for preventing or ameliorating arthritis, in which the health functional food includes a sargahydroquinoic acid as an active ingredient.

The present disclosure relates to a composition for preventing or treating arthritis, in which the composition includes a sargahydroquinoic acid isolated from an extract of Sargassum serratifolium as an active ingredient. The sargahydroquinoic acid has excellent activity to reduce or inhibit the activity of IL-1β, IL-6, or TNF-α, which is an inflammatory cytokine, and has an excellent effect of ameliorating arthritis in an arthritis animal model so that it can be usefully used as a composition for treating arthritis.

In addition, a sargahydroquinoic acid is a natural substance, and has no cytotoxicity, and toxicity to the drug and side effects are avoided, so that it can be safely used even for long-term use, and it is stable in the body.

### BRIEF DESCRIPTION OF DRAWINGS

FIG. 1 is a schematic diagram illustrating a process of isolating a sargahydroquinoic acid from ethanolic (EtOH) extracts of Sargassum serratifolium.

FIG. 2 illustrates the result of measuring the purity of a sargahydroquinoic acid isolated from ethanolic (EtOH) extracts of Sargassum serratifolium.

FIG. 3 is a schematic diagram illustrating a process of preparing an animal model with rheumatoid arthritis.

FIG. 4 illustrates the result of confirming the change in body weight for 7 weeks after administering the sargahydroquinoic acid of the present disclosure to experimental animals with rheumatoid arthritis.

FIG. 5 illustrates the result of confirming the change in the thickness of the paw after administering the sargahydroquinoic acid of the present disclosure to experimental animals with rheumatoid arthritis.

FIG. 6 illustrates the result of confirming the weight of the spleen per body weight after administering the sargahydroquinoic acid of the present disclosure to experimental animals with rheumatoid arthritis and then sacrificing the experimental animals at 6 weeks thereafter.

FIG. 7 illustrates the results of confirming the change of the arthritis index after administering the sargahydroquinoic acid of the present disclosure to experimental animals with rheumatoid arthritis.

FIG. 8 illustrates the result of visually confirming the level of arthritis after administering the sargahydroquinoic acid of the present disclosure to experimental animals with rheumatoid arthritis.

FIG. 9 illustrates the result of confirming the toes taken by micro CT after administering the sargahydroquinoic acid of the present disclosure to experimental animals with rheumatoid arthritis.

FIG. 10 illustrates the result of confirming the joint parts taken by micro CT after administering the sargahydroquinoic acid of the present disclosure to experimental animals with rheumatoid arthritis.

FIG. 11 illustrates the result of quantification of joints taken by micro CT after administering the sargahydroquinoic acid of the present disclosure to experimental animals with rheumatoid arthritis.

FIG. 12 illustrates the result of confirming the level of suppression of the production of an inflammatory cytokine, TNF-α in serum after administering the sargahydroquinoic acid of the present disclosure to experimental animals with rheumatoid arthritis.

FIG. 13 illustrates the result of confirming the level of suppression of the production of an inflammatory cytokine, IL-1β in serum after administering the sargahydroquinoic acid of the present disclosure to experimental animals with rheumatoid arthritis.

FIG. 14 illustrates the result of confirming the level of suppression of the production of an inflammatory cytokine, IL-6 in serum after administering the sargahydroquinoic acid of the present disclosure to experimental animals with rheumatoid arthritis.

### DETAILED DESCRIPTION

The present disclosure relates to a pharmaceutical composition and a health functional food for preventing or treating arthritis, in which the composition includes a sargahydroquinoic acid isolated from an extract of Sargassum serratifolium as an active ingredient.

Sargassum serratifolium is a plant that grows and develops at a range of the intertidal zone to the upper sublittoral belt, which is a perennial large brown algae having the length of 1 to 4 m, has a conical root having the diameter of 4 to 5 cm, is like rubber, and has annual rings. The stem thereof has a columnar structure, is divided into many short central branches, and is differentially pressured. Further, both edges thereof are thin, the stem is vertically raised to one side such as midrib, short branches are at the edges, a stem leaf faces at the base, double saw teeth are at the edges, an air bubble is close to a round shape, and there are follicles or thorn-like protrusions at the top thereof. In particular, it is known to grow and develop on the southern coast of Korea and Jeju Island. It is known to have functions of lipid-lowering, blood pressure lowering, and radioactive isotope release.

The sargahydroquinoic acid may be included in the composition at a concentration of 10 mg/kg to 200 mg/kg, such as 50 mg/kg to 200 mg/kg or 10 mg/kg to 40 mg/kg.

In the present disclosure, “arthritis” refers to degenerative arthritis, rheumatoid arthritis, or lupus arthritis, and for example, it refers to rheumatoid arthritis or degenerative arthritis. The sargahydroquinoic acid of the present disclosure may ameliorate arthritis by reducing or inhibiting the activity of the inflammatory cytokines IL-1β, IL-6 or TNF-α.

As used herein, the term “treating”, unless otherwise indicated, means reversing, alleviating, inhibiting the progress of, or preventing the disorder or condition to which such term applies, or one or more symptoms of such disorder or condition. The term “treatment”, as used herein, refers to the act of treating when “treating” is defined as above. Accordingly, the “treatment” or “therapy” of arthritis in a mammal may include one or more of the following:

(1) inhibiting the development of arthritis,

(2) preventing the spread of arthritis,

(3) reducing arthritis,

(4) preventing recurrence of arthritis, and

(5) palliating symptoms of arthritis

An extract of Sargassum serratifolium according to the present disclosure may be used as those obtained by extracting and isolating from nature using an extraction and separation method known in the art, and the “extract” defined in the present disclosure may be obtained from Sargassum serratifolium using a suitable solvent and include, for example, a crude extract, a polar solvent-soluble extract, or a non-polar solvent-soluble extract of Sargassum serratifolium.

A suitable solvent for extracting an extract from Sargassum serratifolium includes, but is not limited to, an organic solvent. For example, various solvents such as alcohols having 1 to 4 carbon atoms including methanol, ethanol, propanol, isopropanol, butanol, and the like, acetone, ether, benzene, chloroform, ethyl acetate, methylene chloride, hexane, cyclohexane, and the like may be used alone or in combination. Preferably, ethanol (alcohol), n-hexane and ethyl acetate solvents may be used.

As the extraction method, any of methods such as a hot water extraction method, a cold-immersion extraction method, a reflux cooling extraction method, a solvent extraction method, a steam distillation method, an ultrasonic extraction method, a dissolution method, and a compression method can be selected and used. Further, the desired extract may be further subjected to a conventional fractionation process, and may be purified using a conventional purification method. There is no limitation on the production method of the extract of Sargassum serratifolium of the present disclosure, and any known method can be used.

For example, the extract of Sargassum serratifolium is such that a first extract extracted with the solvent extraction method can be prepared in a powder state by an additional process such as vacuum distillation, freeze-drying, or spray-drying. Further, the additional purified fraction can be obtained, in which the first extract is treated using various chromatographs such as silica gel column chromatography, thin layer chromatography, and high-performance liquid chromatography.

Therefore, in the present disclosure, the sargahydroquinoic acid is a concept including all the extracts, fractions and purifications obtained in each step of extraction, fractionation, or purification, diluted solutions thereof, concentrates, or dried products.

The sargahydroquinoic acid of the present disclosure may use the one that is obtained by extraction and isolation from nature using a method of extraction and isolation known in the pertinent field. For example, the sargahydroquinoic acid is isolated from the Sargassum serratifolium alcohol extract through a silica column, and 80% or more of the entire isolated substance includes sargahydroquinoic acid.

The composition of the present disclosure including the sargahydroquinoic acid as an active ingredient may be used as a pharmaceutical composition or a food composition.

The pharmaceutical composition of the present disclosure may be prepared using pharmaceutically acceptable and physiologically acceptable adjuvants in addition to the above-mentioned active ingredients. Excipients, disintegrants, sweeteners, binders, coating agents, swelling agents, lubricants, glidants, or flavoring agents may be used as adjuvants.

The pharmaceutical composition may be formulated into a pharmaceutical composition including at least one pharmaceutically acceptable carrier in addition to the above-described active ingredients for administration.

The pharmaceutical composition may be in the form of granules, powders, tablets, coated tablets, capsules, suppositories, solutions, syrups, juices, suspensions, emulsions, drops, or injectable solutions. For example, for formulation into tablets or capsules, the active ingredient may be combined with an oral, non-toxic pharmaceutically acceptable inert carrier such as ethanol, glycerol, and water. Also, if desired or necessary, suitable binders, lubricants, disintegrants, and coloring agents may also be included as a mixture. Suitable binders include, but are not limited to, natural sugars such as starches, gelatin, glucose, and beta-lactose, natural and synthetic gums such as corn sweeteners, acacia, tragacanth and sodium oleate, sodium stearate, magnesium stearate, sodium benzoate, sodium acetate, and sodium chloride. Disintegrants include, but are not limited to, starch, methylcellulose, agar, bentonite, xanthan gum, and the like. Acceptable pharmaceutical carriers for compositions that are formulated into a liquid solution include saline, sterile water, Ringer's solution, buffered saline, albumin injection solution, dextrose solution, maltodextrin solution, glycerol, ethanol, and a mixture including one or more of these ingredients, which are suitable for the pasteurization and living body. If necessary, other conventional additives such as antioxidants, buffers, and bacteriostatic agents may be added. Further, diluents, dispersants, surfactants, binders and lubricants can be additionally added to formulate into injectable solutions such as aqueous solutions, suspensions, and emulsions, pills, capsules, granules, or tablets. Further, it can be preferably formulated according to each disease or ingredient, using methods disclosed in Remington's Pharmaceutical Science, Mack Publishing Company, Easton Pa., as an appropriate method in the relevant field.

The sargahydroquinoic acid of the present disclosure may be included in an amount of 0.001% to 20% by weight based on the total weight of the composition.

The composition of the present disclosure may also be a food composition. The food composition may include, as an additional ingredient, various flavoring agents or natural carbohydrates such as a conventional food composition, in addition including the sargahydroquinoic acid as an active ingredient.

Examples of the above-mentioned natural carbohydrates include monosaccharides such as glucose and fructose; disaccharides such as maltose and sucrose; and polysaccharides such as conventional sugars, e.g., dextrin and cyclodextrin, and sugar alcohols, e.g., xylitol, sorbitol, and erythritol. The above-mentioned flavoring agents can be advantageously used as natural flavoring agents (thaumatin), stevia extracts (e.g., rebaudioside A and glycyrrhizin) and synthetic flavoring agents (e.g., saccharin and aspartame).

The food composition of the present disclosure can be formulated in the same manner as the pharmaceutical composition and thus used as a functional food or added to various foods. Foods to which the composition of the present disclosure can be added include, for example, beverages, meats, chocolates, foodstuffs, confectionery, pizzas, ramens, other noodles, gums, candies, ice creams, alcoholic beverages, vitamin complexes, and dietary supplement foods.

Further, the food composition may include various additives such as various nutrients, vitamins, minerals (electrolytes), flavoring agents such as synthetic flavors and natural flavors, colorants and enhancers (cheese, chocolate, etc.), pectic acid and its salts, alginic acid and its salts, organic acids, protective colloid thickening agents, pH adjusting agents, stabilizers, preservatives, glycerin, alcohols, carbonating agents used in carbonated drinks in addition to the extract of Sargassum serratifolium as an active ingredient. Further, the food composition of the present disclosure may include fruit flesh for producing natural fruit juice, and fruit juice drinks and vegetable drinks.

Since the sargahydroquinoic acid, which is an active ingredient of the present disclosure, is a natural substance and has little side effects of chemicals, it can be safely used for even long-term administration for the purpose of prevention and treatment of arthritis.

The present disclosure also provides a health functional food for ameliorating arthritis, in which the health functional food includes a sargahydroquinoic acid as an active ingredient.

The health functional food of the present disclosure can be manufactured and processed in the form of tablets, capsules, powders, granules, liquids, pills, etc. for the purpose of amelioration of arthritis.

In the present disclosure, the term “health functional food” refers to foods prepared and processed using raw materials or ingredients having useful functions in accordance with Korean Health Functional Foods Act, No. 6727, which means that it is ingested for the purpose of obtaining a beneficial effect for health use such as control of nutrients or physiological action for the structure and function of the human body.

The health functional food of the present disclosure may include conventional food additives and, unless otherwise specified, whether or not they are suitable as food additives is determined by standard and criteria on corresponding products according to the General Rules and General Test for Korean Food Additives Codex approved by the Korean Ministry of Food and Drug Safety.

Examples of the items listed in the above-mentioned “Food Additives Codex” include chemical compounds such as ketone, glycine, calcium citrate, nicotinic acid, and cinnamic acid; natural additives such as persimmon color, licorice extract, crystalline cellulose, kaoliang color, and guar gum; mixture preparations such as L-glutamic acid sodium preparations, alkali agents for noodles, preservative preparations, and tar coloring preparations.

For example, the extract of Sargassum serratifolium, an active ingredient of the present disclosure, is mixed with the excipient, binder, disintegrant, and other additives, then the mixture is granulated in a conventional manner, and then a lubricant and the like is added and the granulate is compression-molded, or then the mixture can be directly compression-molded, thereby obtaining the health functional food in the form of tablet. In addition, the health functional food in the tablet form may include a flavoring agent or the like as needed.

The hard capsule of the capsule-type health functional food can be prepared by filling a conventional hard capsule with a mixture of the extract of Sargassum serratifolium, an active ingredient of the present disclosure, with an additive such as an excipient. The soft capsule may be prepared by filling a capsule base such as gelatin with a mixture of the extract of Sargassum serratifolium, an active ingredient of the present disclosure, with an additive such as an excipient. The soft capsule may include plasticizers such as glycerin and sorbitol, coloring agents, preservatives and the like, if necessary.

The pill-type health functional food can be prepared by molding a mixture of the sargahydroquinoic acid, an active ingredient of the present disclosure with excipients, binders, disintegrants, and the like, according to a known method. If necessary, it may be coated with white sugars or other coating aids, or the surface thereof may be coated with a material such as starch and talc.

The granule-type health functional food can be prepared by a conventionally known method in which a mixture of the sargahydroquinoic acid, an active ingredient of the present disclosure with excipients, binders, and disintegrants is formed into granules. If necessary, fragrance agents, flavoring agents, etc. can be added.

The health functional food may be a beverage, a meat, a chocolate, a foodstuff, a confectionery, a pizza, a ramen, a noodle, a gum, a candy, an ice cream, an alcoholic beverage, a vitamin complex, a health supplement food, etc.

The present disclosure also provides a method for preventing or treating arthritis, which includes administering the sargahydroquinoic acid to a mammal.

As used herein, the term “mammal” refers to a mammal that is the subject of treatment, observation, or experiment, preferably a human

As used herein, the term “therapeutically effective amount” means that the amount of the active ingredient or the pharmaceutical composition sufficient to induce biological or medical responses in tissues, animals or human evaluated by researchers, veterinarians, medical doctors or clinical doctors, including the amount sufficient to alleviate diseases or disorders to be treated. It is obvious to one skilled in the art that an appropriate administration dosage and the administration number for active ingredients of the present disclosure may be changed depending on effect to be required. Therefore, an appropriate administration dosage may be determined by one skilled in the art and regulated variously depending on factors such as type of disease, severity of disease, content of active ingredients and other ingredients contained in composition, type of formulation, age of a patient, body weight of a patient, health state of a patient, gender of a patient, diet of a patient, administration time, administration route, excretion rate of composition, administration period, drug used simultaneously. In the treatment method of the present disclosure, preferably, a dosage of the extract of Sargassum serratifolium of the present disclosure may be 0.01 mg/kg to 100 mg/kg once or several times a day in case of an adult.

In the treatment method of the present disclosure, the extract of Sargassum serratifolium of the present disclosure may be administrated in conventional manners through oral, rectal, intravenous, arterial, intraperitoneal, intramuscular, intrasternum, transdermal, topical, intraocular, or intradermal route.

Hereinafter, the present disclosure will be described in detail with reference to exemplary embodiments. It would be obvious to those skilled in the art that these exemplary embodiments are for specifically illustrating the present disclosure and the scope of the present disclosure is not limited to these exemplary embodiments.

### EXAMPLE 1

Isolation of sargahydroquinoic acid (SHQA)

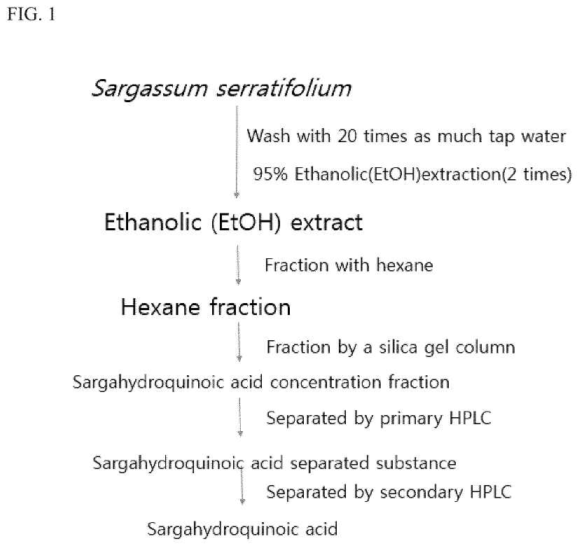

S. serratifolium was collected in May 2017 from the Tongyeong coast in Korea. Specimens were confirmed by C. G. Choi, an algae taxonomist at Department of Ecology of Pukyong National University. The fresh S. serratifolium was dried naturally and then stored frozen until further extraction. The isolation process of a sargahydroquinoic acid from S. serratifolium is as illustrated in FIG. 1. The dried S. serratifolium (2.0 kg) was immersed in 20 times as much tap water for 2 hours and then extracted twice at 70° C. for 6 hours with 95% (v/v) ethyl alcohol (8 L/each). The extracts were mixed and then filtered using ultrafiltration unit (5 kDa). The filtrate was concentrated to a rotary evaporator (Eyela N-3010) equipped with a vacuum controller (NVC-2200) and a cooling device (CA-2600). During concentration of the extract, the salt and the water-soluble saccharides were removed by repeated solubilization of the lipophilic fraction and finally fraction rich in meroterpenoid (160 g) was obtained. For further fractionation of the extract, the extract was suspended in H₂O:ethanol (9:1, v/v) and partitioned into n-hexane (120 g), ethyl acetate, n-butanol, and water. An aliquot of n-hexane fraction was dissolved in dichloromethane and then eluted stepwise with a mixture of 0.2%, 2.0% and 5.0% methanol in CH₂Cl₂ using silica gel (70-230 mesh, Merck) column (10×100 cm). The fraction eluted with 0.2% methanol in CH₂Cl₂ showed the strongest anti-inflammatory activity and contained a high concentration of a sargahydroquinoic acid. In order to purify the sargahydroquinoic acid, 20 g of the silica gel fraction was dissolved in 200 ml of methanol, filtered through a membrane filter, and then 200 μL was injected and separated by HPLC equipped with a Phenomenex C18 (2) (15 μm, 21.2 mm×250 mm) column. As a mobile phase of the chromatography, a mixed solvent of methanol (A) and water (B) was used and the flow rate was set to 7 mL. The chromatographic conditions consisted of linear gradient at first A/B (85/15) to A/B (95/5) concentration for 30 minutes, A/B (95/5) to A/B (100/0) concentration for 2 minutes and a re-equilibration at A/B (78/22) for 10 minutes after being washed at A/B (100/0) for 10 minutes. Chromatography was carried out 100 times by autosampler injection of samples automatically to obtain a large amount of a sargahydroquinoic acid. In order to increase the purity of the bulk sargahydroquinoic acid, Phenomenex C18 (2) (5 μm, 10 mm×250 mm) column was used in the same apparatus. The chromatographic conditions consisted of linear gradient at first A/B (78/22) to A/B (87/13) concentration for 20 minutes, A/B (87/13) to A/B (100/0) concentration for 2 minutes and a re-equilibration at A/B (78/22) for 10 minutes after being washed at A/B (100/0) for 6 minutes. Chromatography was carried out by autosampler injection of samples automatically to obtain higher purity of a sargahydroquinoic acid.

Analysis of sargahydroquinoic acid content

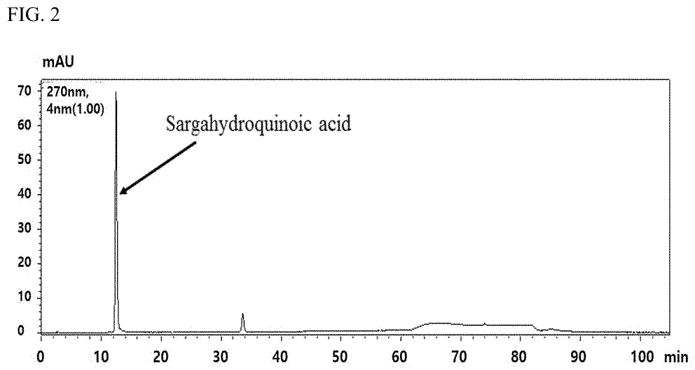

An aliquot of 0.2% methanol fraction was separated from the silica gel column by an HPLC system equipped with a Phenomenex Luna RP-18 (2) column (3 μm, 3.0×150 mm, Phenomenex, Torrence, Calif., USA). The mobile phase consisted of 0.1% formic acid (A) in methanol and 0.1% formic acid (B) in water. In order to analyze the purity of a sargahydroquinoic acid by HPLC, the elution conditions consisted of 2 minutes linear gradient from A/B (78/22) to A/B (95/5) for 88 minutes, A/B (95/5) to A/B (100/0) and a re-equilibration for 10 minutes at A/B (78/22). The flow rate was 0.34 mL/min and the injection volume was 3 μL. The purity of a sargahydroquinoic acid was measured to be about 95% or higher (FIG. 2).

### EXAMPLE 2

Therapeutic effect of a sargahydroquinoic acid on rheumatoid arthritis

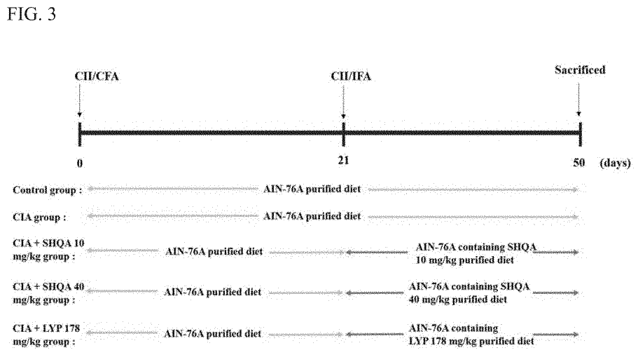

Eight weeks old male DBA/1J mice (Central Lab Animal Inc., Seoul, Korea) were raised for 1 week in an environment maintaining temperature (25±2° C.), humidity (55%) and 12 hour light and dark cycle. All experiments were carried out in accordance with the guidelines of the Korean National Institutes of Health Guide for the Care and Use of Laboratory Animals, and the animal testing procedure was approved by the Animal Ethics Committee of Pukyong National University (2015-12). Rheumatoid arthritis was induced in male DBA/1J mice. Type II collagen was emulsified (CFA; 2 mg/mL) in the same volume of complete Freund's adjuvant. On day 0, 100 μg of CFA was injected intradermally into the lower tail portion of the DBA/1J mice to immunize. After 21 days, 100 μg of CII (IFA; 2 mg/mL) emulsified in the same volume of incomplete Freund's adjuvant in the lower tail portion of the mice was boosted by intradermal injection. On day 42, all mice were anesthetized with ether and sacrificed. Blood (plasma sample), spleen, liver and joint tissues were immediately removed and stored at −80° C. In order to assess the effect of a sargahydroquinoic acid (SHQA) on rheumatoid arthritis of CIA mice, DBA/1J mice were randomly assigned to four groups (n=10 per group): a control group (an untreated group), a CIA induced arthritic group, and CIA induced and SHQA-treated groups. AIN-76A purified diet was provided to both the control and CIA groups. CIA-induced and SHQA-treated mice received AIN-76A containing 10.0 mg SHQA/kg and 40 mg SHQA kg, respectively. All mice were free to access food and water for 42 days (FIG. 3).

<2-1> Measurement of Body Weight Change According to Treatment of Sargahydroquinoic Acid

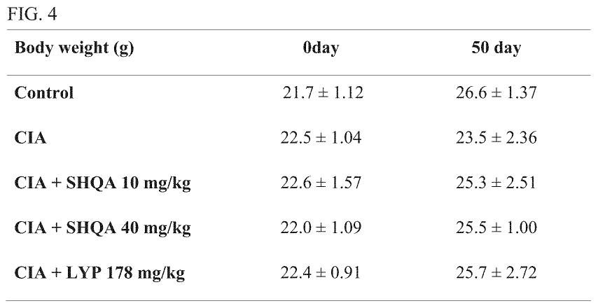

For the control group without treatment, the arthritis animal group (CIA treatment group), and the treated group in which arthritis experimental animals were treated with 10 mg and 20 mg of SHQA, their body weights were measured weekly for 7 weeks. As a result, there was no significant change in body weight per group. (FIG. 4).

<2-2> Measurement of Change of Paw Thickness According to Treatment of Sargahydroquinoic Acid

The present inventors measured the change in paw thickness, which is an index of arthritis, in order to confirm the therapeutic effect of rheumatoid arthritis, caused by the SHQA.

The change in paw thickness according to the treatment with the SHQA every 5 days was measured using an electronic caliper for 25 days after the second subcutaneous injection in the preparation process of the experimental animal in which rheumatoid arthritis was induced in <2-1>.

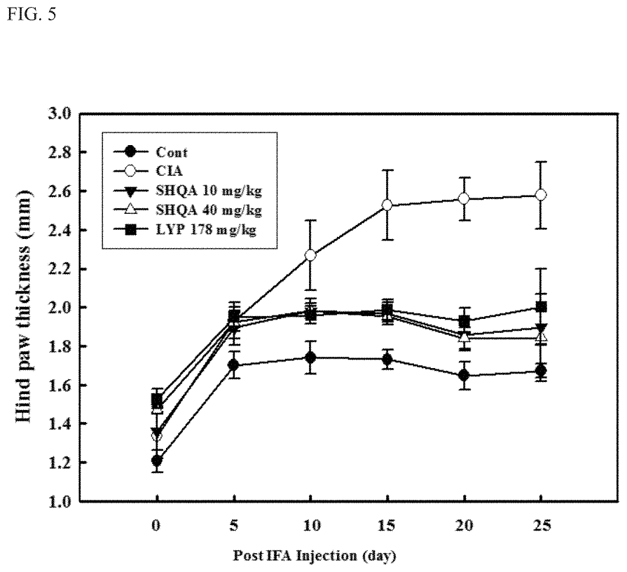

As a result, it was found that the paw thickness was increased by inflammation in the experimental animals in which arthritis was induced, whereas the paw thickness was significantly decreased in the group treated with the SHQA, and it was confirmed that the thickness thereof after 25 days was similar to that of the control group (FIG. 5).

<2-3> Weighing of Spleen per Body Weight According to Treatment of Sargahydroquinoic Acid

The present inventors measured the weight of the spleen in order to determine whether or not the SHQA can treat rheumatoid arthritis.

A spleen is the major organ of the human immune system, and relative weights are often used as a preliminary indicator to assess the immunoregulatory activity of the sample to be tested.

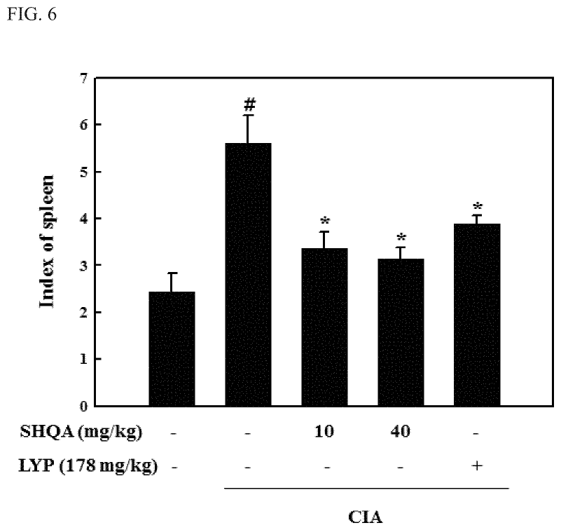

As a result of measuring the weight of the spleen per body weight, the splenoma was found (spleen was swelled and enlarged) in the arthritis-induced experimental animal group, but no splenoma appeared in the group treated with the SHQA of the present disclosure. Thus, it was confirmed that the expansion of the spleen and permeability of immune cells were inhibited by the SHQA (FIG. 6).

<2-4> Measurement of Arthritic Index According to Treatment of Sargahydroquinoic Acid

The present inventors observed the arthritis index by visual inspection in order to confirm the therapeutic effect of the SHQA on rheumatoid arthritis, and the arthritis index of each was scored by 0 to 4 points and measured by five non-experts.

The arthritis index was calculated by summing the scores for the respective paws per one number of mouse (highest score =16 points).

—Evaluation Standard—

0 point: there is no symptom;

0.5 point: one finger is red and swelled;

1 point: 2 to 5 fingers are red and a little swelled;

2 points: 2 to 5 joints are red and moderately swelled;

3 points: whole paws are red and swelled; and

4 points: swelling and malformation are reduced.

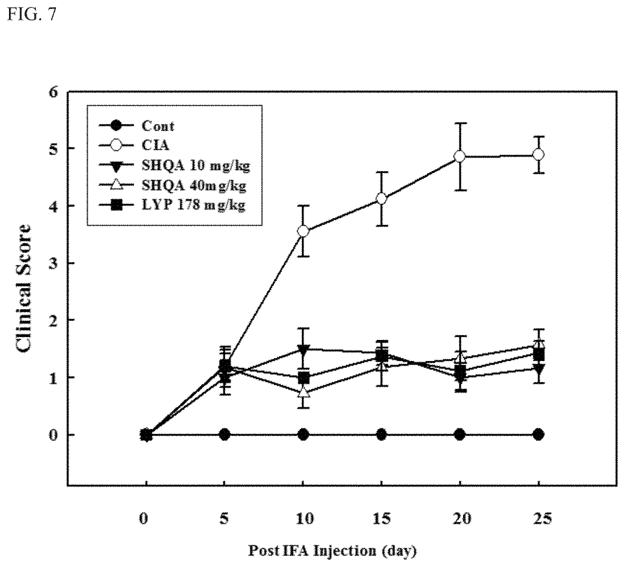

As a result of evaluating the rheumatoid arthritis score of each treatment group, it was found that the arthritis index was high in the arthritis-induced experimental animal group, but the arthritis evaluation index was significantly decreased in the treatment group with which the SHQA was treated (FIG. 7).

<2-5> Visual Observation of Level of Arthritis Treatment According to Treatment of Sargahydroquinoic Acid

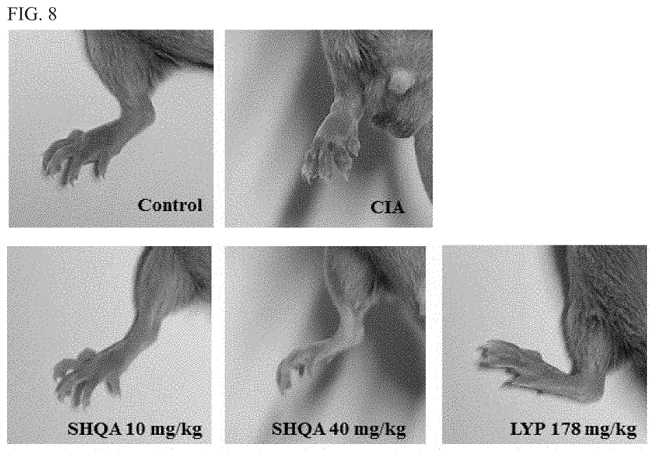

The present inventors observed visually the level of arthritis treatment according to treatment of the SHQA. As a result, the hind paw was severely swollen and erythema symptoms were showed in the arthritis-induced experimental animal group. However, it was confirmed that swelling was reduced and erythema symptoms were alleviated in the treatment groups treated with the SHQA of the present disclosure with 10 mg/kg and 40 mg/kg for 25 days (FIG. 8).

<2-6> Effect of Micro-CT Bone Associated Index Change According to Treatment of Sargahydroquinoic Acid

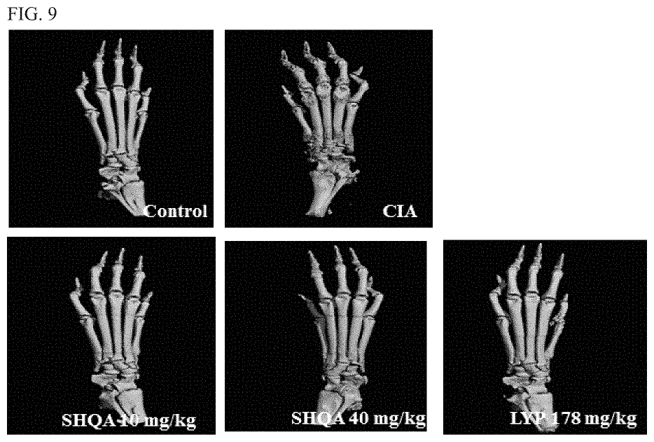

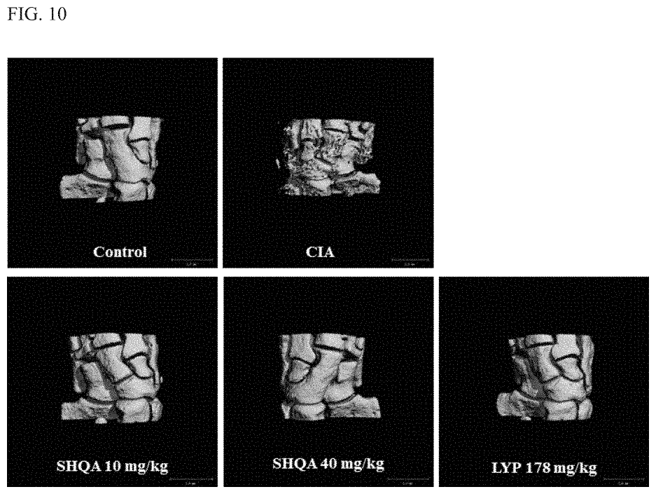

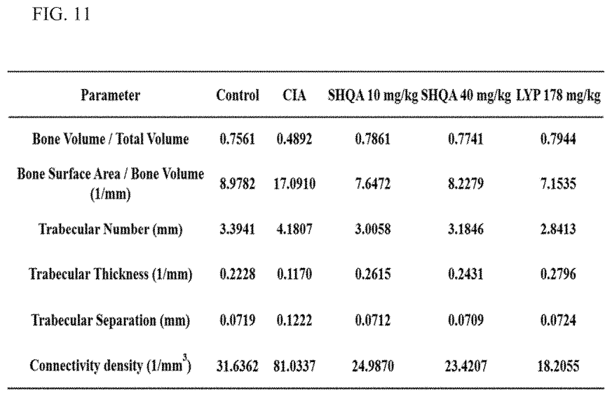

Computed tomography of the joints was performed using a SKYScan 1172 scanner (Bruker, Belgium, Va., USA). Three-dimensional microstructure image data was reconstructed by calculating structural indexes using CTAn software (Bruker, Belgium). Joint cracks were observed in the arthritis-induced experimental animal group, but bone and joint cracks were not observed in the treatment group treated with SHQA at 10 mg/kg and 40 mg/kg for 25 days and in the group treated with lyprinol (FIGS. 9 and 10). In addition, bone mineral density, which is an index of bone health, was improved in the SHQA and lyprinol groups compared to the CIA group, and the volume-to-surface area of the bones was also significantly decreased (FIG. 11).

<2-7> Inhibitory Effect of Inflammatory Cytokine According to Treatment of Extract of Sargassum serratifolium

When rheumatoid arthritis develops in general, inflammatory mediators such as interleukin (IL) and PGE₂ are released into joints and blood by activated immune cells gathered at inflammatory sites. In particular, TNF-α, IL-6, and IL-1β can be used as an index to determine the level of disease of rheumatoid arthritis.

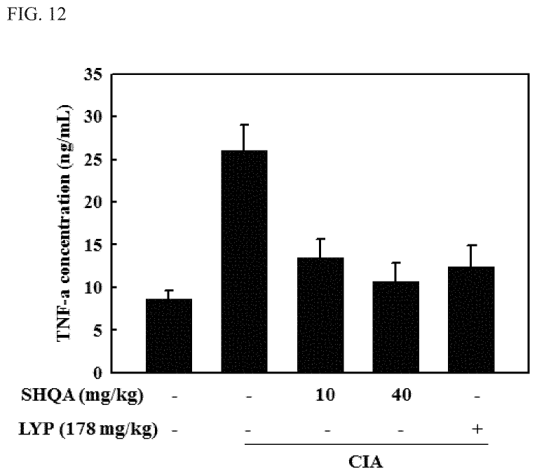

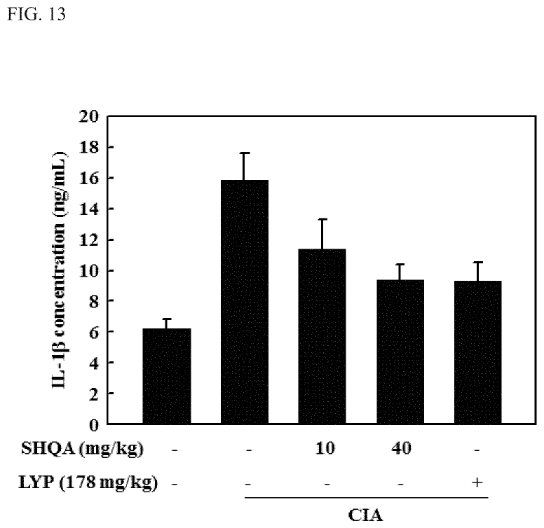

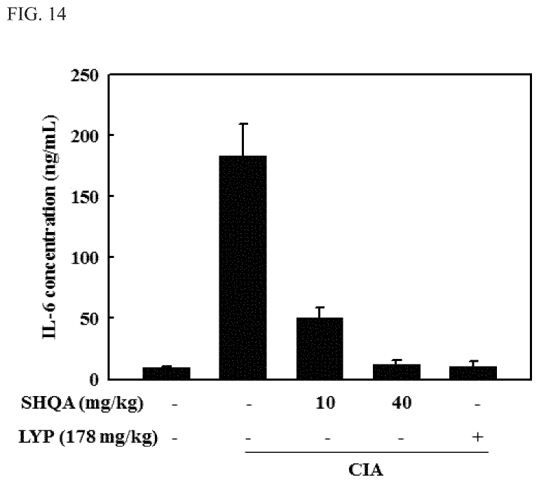

The present inventors measured the expression level of the inflammatory cytokine in the blood of an experimental animal using an ELISA kit. As a result, it was confirmed that the inflammatory cytokine rapidly increased in the arthritis-induced experimental animal group, but the expression of inflammatory cytokines in TNF-α (FIG. 12), IL-1β (FIG. 13) and IL-6 (FIG. 14) was significantly inhibited in the group treated with the SHQA of the present disclosure for 25 days.

Hereinbefore, the present disclosure has been described with reference to the preferred embodiments. It will be understood by those skilled in the art that the present disclosure may be embodied in various other forms without departing from the essential characteristics thereof. Therefore, the disclosed embodiments should be considered in an illustrative rather than a restrictive perspective. The scope of the present disclosure is defined by the appended claims rather than by the preceding description, and all differences within the scope of equivalents thereof should be construed as being included in the present disclosure.

What is claimed is:

1. A method of preventing or treating arthritis, the method comprising administering an effective amount of a sargahydroquinoic acid compound or salt thereof to a subject in need thereof.

2. The method of claim 1, wherein the sargahydroquinoic acid is obtained from an extract of Sargassum serratifolium using an organic solvent.

3. The method of claim 2, wherein the organic solvent is selected from the group consisting of methanol, ethanol, propanol, isopropanol, butanol, acetone, ether, benzene, chloroform, ethyl acetate, methylene chloride, hexane, and cyclohexane.

4. The method of claim 1, wherein the sargahydroquinoic acid reduces or inhibits the activity of IL-1β, IL-6, or TNF-α, which is an inflammatory cytokine.

5. The method of claim 1, wherein the arthritis is degenerative arthritis, rheumatoid arthritis, or Lupus arthritis.

6. A health functional food for preventing or treating arthritis, the health functional food comprising a sargahydroquinoic acid as an active ingredient.
